(19) United States
(12) Patent Application Publication
(10) Pub. No.: US 2002/0173014 A1
(43) Pub. Date: Nov. 21, 2002

(54) Method to control cellular (3R)-hydroxyacyl-CoA esters, precursor molecules for polyhydroxyalkanoate synthesis in genetically modified organisms

(76) Inventors: Kalervo Hiltunen, Oulu (FI); Tuomo Glumoff, Oulu (FI)

Correspondence Address: BIRCH STEWART KOLASCH & BIRCH, PO BOX 747, FALLS CHURCH, VA, 22040-0747, US

(21) Appl. No.: 10/060,230

(22) Filed: Feb. 1, 2002

Related U.S. Application Data
(63) Continuation-in-part of application No. PCT/FI00/00663, filed on Aug. 2, 2000.

(30) Foreign Application Priority Data
Aug. 3, 1999 (FI) 19991667

(51) Int. Cl. C12P 7/62; C12N 9/16; C12N 15/87

(52) U.S. Cl. 435/135; 435/455; 435/196

(57) ABSTRACT

This invention relates to a method for controlling the production of cellular (3R)-hydroxyacyl CoA esters of predetermined length in a chosen host cell or organism. In particular, the invention relates to a modified gene encoding a multifunctional 2-enoyl-CoA hydratase 2/(3R)-hydroxyacyl CoA dehydrogenase enzyme type 2 protein and DNA constructs and host comprising the gene. The modification of the gene results in the enrichment of cellular (3R)-hydroxyacyl CoA esters of predetermined length, when the gene is introduced and expressed in a host cell oxidizing exogenous or endogenous β-fatty acids.

### FIELD OF THE INVENTION

This invention relates to a method for controlling the production of cellular (3R)-hydroxyacyl CoA esters of predetermined length in a host cell or organism and in particular to a method for producing polyhydroxyalkanoates (PHAs). This invention relates also to a method for preparing a host cell or organism capable of producing PHAs and to the host cell or organism prepared by the method. Furthermore the present invention relates to a modified gene encoding a multifunctional 2-enoyl-CoA hydratase 2/(3R)-hydroxyacyl CoA dehydrogenase enzyme type 2 protein and to the enzyme encoded by the gene as well as DNA constructs, vectors and hosts comprising the gene.

### BACKGROUND OF THE INVENTION

In a cell, (3R)-hydroxyacyl metabolites are intermediates of lipid metabolism in both biosynthetic and catabolic processes. Examples of biosynthetic events are the formation of polyhydroxyalkanoates (PHAs) (Donadio et al., 1991; Poirier et al., 1995) and the de novo synthesis of fatty acids. Polyhydroxyalkanoate synthetases in microbes use (3R)-hydroxyacyl-CoA thioesters as substrates, whereas fatty acid synthetases have acyl carrier protein (ACP) as a carrier for acyl groups. Concerning the catabolic processes, if peroxisomal β-oxidation of fatty acids proceeds via multifunctional 2-enoyl-CoA hydratase 2/(3R)-hydroxyacyl-CoA dehydrogenase enzyme type 2 (MFE-2), it occurs via (3R)-hydroxyacyl-CoA intermediates (Hiltunen el al., 1992; Dieuaide-Noubhani, M. et al., 1996; Qin et al., 1997b). The (MFE-2)-dependent pathway operates in yeast peroxisomes, whereas, additionally, a pathway depending on a multifunctional 2-enoyl-CoA hydratase 1/enoyl-CoA isomerase/(3S)-hydroxyacyl-CoA dehydrogenase enzyme type 1 (MFE-1), which utilizes (3S)-hydroxyacyl-CoA intermediates similarly to mitochondrial and bacterial β-oxidation, is also found in mammalian peroxisomes.

FIG. 1 depicts the joining of the β-oxidation of fatty acids with the biosynthesis of polyhydroxyalkanoates. Enzymes essential for PHA-synthesis in the reaction pathway are multifunctional enzyme type 2 (MFE-2) and PHA-synthetase. The hydratase-2 domain of MFE-2 (MFE-2-h) produces the substrate for PHA-synthetase after the dehydrogenase activity of MFE-2 (MFE-2-d) has been modified. The branch utilizing the (S)-isomer of the substrate is needed for energy production. On the right-hand side of the figure the PHA-synthesis pathway is presented as it functions in bacteria.

This far MFE-2 has been cloned and identified in a large variety of eukaryotes—among others yeast and several mammalian species including man—but not in prokaryotes. All identified enzymes are chimeric multifunctional proteins with an N-terminal domain belonging to the short chain alcohol dehydrogenase/reductase superfamily.

The yeast enzyme has two dehydrogenase-like domains but it was previously not known whether they both are active or play a role in the metabolism. The dehydrogenase domain is followed by a 2-enoyl-CoA hydratase 2 domain which has been shown to catalyze hydratation/dehydration of trans-2-enoyl-CoA and (3R)-hydroxyacyl-CoA esters in a reversible manner. The mammalian enzymes, but not the yeast ones, have an additional C-terminal sterol carrier protein 2-like domain, the physiological function of which is unclear.

A large number of both gram negative and positive bacteria can synthesize PHA from suitable acyl-CoA building blocks by the enzyme PHA-synthetase. Although PHAs accumulating as inclusions in bacterial cytoplasm are thermoresistant and water insoluble polymers, they are completely decomposed by microorganisms. Therefore, as a source of inherently biodegradable plastics and elastomers, the PHAs have attracted a wide biotechnological interest.

Although the main commercial value of PHAs is in their potential applications as biodegradable plastics, the epimers of monomeric 3-hydroxyacids with any chain length can be used as reagents in biomedical research. However, except (3S)- and (3R)-hydroxybutyric acids, the pure epimers of 3-hydroxy fatty acids are currently obtained via laborious organic synthesis. Therefore, they are costly and their availability is limited.

The best known and studied PHA is 3-polyhydroxybutyrate (PHB). All the carbon atoms of PHBs originate from acetyl-CoA. First two acetyl-CoA molecules are condensed by the reversal of 3-ketoacyl-CoA thiolase reaction. The formed acetoacetyl-CoA is subsequently reduced by an NADPH-dependent acetoacetyl-CoA reductase to (3R)-hydroxybutyryl-CoA, which serves as a substrate for polyhydroxyacyl-CoA synthetase(s). Commonly, carbohydrates such as glucose can serve as carbon sources to generate acetyl-CoA. If the carbon chain length of the monomeric unit exceeds five carbons, the acyl moieties are thought to arise from β-oxidation via trans-2-enoyl-CoA, (3S)-hydroxyacyl-CoA and 3-ketoacyl-CoA intermediates coupled to the reaction catalyzed by 3-ketoacyl-CoA reductase. There is evidence suggesting that the building blocks of PHAs can also be channelled from intermediates of the de novo synthesis pathway of fatty acids. In this pathway acyl carrier protein (ACP) acts as acyl group carrier but as PHA synthetase(s) use hydroxyacyl-CoA esters as substrates the complete molecular mechanism of acyl transfer is unclear.

The increasing demand for biodegradable materials has led to the development of, among other materials, biodegradable plastics. Already in 1995 there were some twelve inherently biodegradable plastics on the market (Poirier et al., 1995) including PHAs. The physical properties of PHAs vary depending on the chemical units they are composed of and they can be stiff and brittle or elastic and rubber-like. Polyhydroxybutyrate (PHB) is an example of the former type; it consists of four-carbon units and so is a polymer of short-chain units. Over 40 different PHAs have been characterized (Steinbüchel, 1991).

PHAs can be produced by bacterial fermentation. Different types of PHAs are produced depending on the carbon source and bacterial strain used. Most bacteria studied can only synthesize either short-chain PHAs (three to five carbons long units; C3-C5) or medium-chain PHAs (C6-C14), with only a few capable of synthesizing both types (Poirier et al., 1995). Yao et al. (1999) describes a Pseudomonas nitroreducens-strain capable of synthesizing PHB or medium chain length PHAs by changing the fatty acid substrate composition. However, it is more expensive to produce PHAs in this way than to chemically synthesize plastics of comparable properties (Poirier et al., 1995). Even the production of PHB by bacteria, which requires only glucose as carbon source, is too costly, not to mention the production of C6-C14 PHAs, for which fatty acids are needed—and these are even more expensive than glucose. A further disadvantage is that such synthesis will result in a pool of PHAs consisting of building blocks of ca. 6 to 16 carbons. This fact, added to the inevitable losses in the purification of PHAs with defined chain lenghts, will diminish the relative yields and increase the costs per unit produced (Poirier et al., 1995).

Therefore, plants have been examined as new production hosts for PHAs, because of their high capacity of production and efficient methods for harvesting. The first experiment included the expression of the PHB biosynthetic genes of Alcaligenes eutrophus in Arabidopsis thaliana to produce 100 μg of PHB per g of fresh plant (Poirier et al., 1992). Later the yield could be increased 100-fold by targeting the PHB to plastids instead of cytoplasm; PHB then constituted 14% of the dry weight of the harvested plant (Nawrath et al., 1994). More recently, C6-C16 PHAs were shown to be synthesized by A. thaliana when the PHA synthetase was targeted to peroxisomes and it could use intermediates of the peroxisomal fatty acid β-oxidation as source for PHA (Mittendorf et al., 1998). However, the molecular weights of the PHAs were lower than those of PHAs produced by bacteria. This may be due to the excessive activity of PHA synthetase compared with the substrate available to the enzyme.

Some companies are studying PHA production in oil crop plants, one experimenting with rapeseed (Zeneca Seeds (UK)), another working with both rapeseed and soybean (Monsanto (USA)). The costs of producing PHAs depend on many factors, such as the absolute amount of PHA produced, the effect of PHA on the starch, lipid and protein contents of the plants and the extraction of the PHA from the plant tissues (Poirier et al., 1995).

WO-A-9935278 suggests the transformation of plants by a polyhydroxylalkanoate synthase gene fused nucleotide sequence encoding peptide required for peroxisomal targeting peroxisome. WO-A-99/45122 suggests the modification of fatty acid biosynthesis and oxidation in plants to make new polymers by using hydratases and β-oxidation enzyme system.

The production of PHB via fermentation technology using glucose as a carbon source makes the end product expensive (Gerngross, 1999) and the physical properties of PHB restrict its use as a biodegradable plastic. The transgenic plants are attractive alternatives for PHA production. However, independent of the hosts used as bioreactors, the production of PHAs with specified monomer chain lenghts other than C4-C5 is not controllable by the prior art technology.

### SUMMARY OF THE INVENTION

It is an aim of the present invention to eliminate the problems associated with the prior art and to provide a method to control the production of, polyhydroxyalkanoates or other type of polymers containing (3R)-hydroxyacyl groups and their derivates (here called PHAs) in a chosen production host cell or organism.

Furthermore, it is an aim of the present invention to provide a method to produce PHAs consisting of building blocks of predetermined chain lenghts in a host cell or organism.

The present invention is based on the finding that the cellular (3R)-hydroxyacyl pool can be controlled by genetic means in a host cell or organism. According to the present invention it is possible to genetically change the substrate specificity of the multifunctional 2-enoyl-CoA hydratase 2/(3R)-hydroxyacyl CoA dehydrogenase enzyme type 2 (MFE-2) protein and by this way to control the chain lengths of the (3R)-hydroxyacyl-CoA intermediates in the cellular (3R)-hydroxyacyl pool. PHA synthetase present in the production host uses the (3R)-hydroxyacyl-CoA intermediates of desired chain lengths to synthesize PHAs with desired chain lengths and desired properties. Many bacteria are capable of producing PHA synthetase, and if desired, this capability can be transferred into a yeast, or other fungus, into a higher eucaryote, preferably into a plant.

In the research work forming the basis of the invention it could be shown that the dehydrogenase domain or dehydrogenase domains and hydratase domain have different substrate specificities. The protein encoded by yeast MFE type 2 gene, having two copies of the dehydrogenase-domain, processes different chain-length fatty acids with domain A than it does with domain B: domain A has its highest catalytic activity towards medium and long chain-length 2-enoyl-CoA, while domain B has a broader substrate specificity with the highest turn-over rate with short chain substrates. This was found out by inactivating either of the domains, or both, and testing the purified enzyme activities for β-oxidation of various fatty acids (in vitro test) as well as the growth of yeast on fatty acids as sole carbon source (in vivo test).

According to one embodiment of this invention the gene encoding MFE-2 protein is modified by genetically altering the gene region encoding the dehydrogenase domain or domains. If the domain responsible for the oxidation of medium and long chain-length 2-enoyl-CoA is inactivated and the gene is expressed in a chosen host, this results in the accumulation of medium and long chain-length 2-enoyl-CoA. In contrast, when the domain responsible for the oxidation of short chain-length 2-enoyl-CoA is inactivated, this results in the accumulation of short chain-length 2-enoyl-CoA.

According to one preferred embodiment the gene encoding a MFE-2 protein that is genetically modified to alter the region encoding the dehydrogenase domain or domains, wherein the gene is preferably a non-human gene. In another embodiment of the invention, the MFE-2 gene may be a human gene which has been modified with any amino acid modification, except for replacing the glycine at the 16^{th} amino acid residue with a serine.

According to another preferred embodiment of the invention the MFE-2 encoding gene originates from yeast and comprises two gene regions encoding dehydrogenase domains and one gene region encoding a hydratase domain. If the yeast domain having the highest catalytic activity towards medium and long chain-length 2-enoyl-CoA is inactivated and the gene is expressed in a chosen host, this results in the accumulation of about C8-C12 chain-length 2-enoyl-CoA. In contrast, when the yeast domain having the highest turn-over rate with short chain substrates is inactivated, this results in the accumulation of about C6-C8 chain-length 2-enoyl-CoA.

If in the chosen gene there is only one dehydrogenase domain, the hydratase domain is responsible for the (3R)-hydroxyacyl metabolites. If the human dehydrogenase domain is inactivated in the gene encoding the human MFE-2 and the gene is expressed in a chosen host, this results in the control of the (3R)-hydroxyacyl intermediate pool by the kinetic properties of the hydratase 2 domain. In the case of the mammalian enzyme, mainly medium chain and to certain extend longer (3R)-hydroxyacyl metabolites will be synthesized.

The present invention thus gives a possibility to control the synthesis of the cellular (3R)-hydroxyacyl metabolites and to direct the synthesis to intermediates of desired chain lengths. The gene to be modified originates preferably from yeast, other fungus or from mammal. In the yeast MFE-2 gene one of the two dehydrogenases may be inactivated or both dehydrogenases may be inactivated. If the MFE-2 gene is of mammalian origin one dehydrogenase domain may be inactivated.

One object of this invention to provide a method for controlling the production of cellular (3R)-hydroxyacyl CoA esters of predetermined length in a host cell or organism, which method comprises the steps of:

introducing a gene encoding a multifunctional 2-enoyl-CoA hydratase 2/(3R)-hydroxyacyl CoA dehydrogenase enzyme type 2 protein (MFE-2) comprising at least one gene region encoding a hydratase domain and at least one gene region encoding a dehydrogenase domain, and wherein at least one genetic change has been made to the gene region encoding dehydrogenase domain, resulting in the enrichment of cellular (3R)-hydroxyacyl CoA esters of predetermined length, when the gene is introduced and expressed in a host cell oxidizing exogenous or endogenous β-fatty acids, or a DNA construct or a vector comprising the gene, into a host cell or organism; and

growing the host cell or organism under suitable growth conditions in the presence of a carbon source comprising fatty acids from an endogenous source or originating from exogenous additions resulting in the enrichment of cellular (3R)-hydroxyacyl CoA esters of predetermined length.

More specifically, the method according to the invention is mainly characterized by what is stated in the characterizing part of claim 1.

Another object of this invention is to provide a method for preparing a host cell or organism capable of producing PHAs, said host cell or organism expressing an endogenous or foreign gene or genes encoding (3R)-hydroxyacyl-CoA ester polymerizing enzyme or enzymes, which method comprises the steps of:

introducing a gene encoding a multifunctional 2-enoyl-CoA hydratase 2/(3R)-hydroxyacyl CoA dehydrogenase enzyme type 2 protein (MFE-2), which comprises at least one gene region encoding a hydratase domain and at least one gene region encoding a dehydrogenase domain, wherein at least one genetic change has been made to the gene region encoding dehydrogenase domain, resulting in the enrichment of cellular (3R)-hydroxyacyl CoA esters of predetermined length, when the gene is introduced and expressed in a host cell oxidizing exogenous or endogenous fatty acids, or a DNA construct or a vector comprising the gene, into the host cell or organism; and

growing the host cell or organism under suitable growth conditions in the presence of a carbon source comprising fatty acids from an endogenous source or originating from exogenous additions.

More specifically, the method according to the invention is mainly characterized by what is stated in the characterizing part of claim 2.

A third object of this invention is to provide a method for producing PHAs in an organism or organisms expressing endogenous or foreign gene or genes encoding (3R)-hydroxyacyl-CoA esters polymerizing enzyme or enzymes, which method comprises the steps of:

introducing a modified MFE-2 encoding gene, or DNA-construct or vector comprising the gene into a host cell or organism;

growing the host cell or organism under suitable growth conditions in the presence of a carbon source comprising fatty acids originating from endogenous source or exogenous additions;

recovering PHAs or their hydrolysis products from the host cells or organisms.

More specifically, the method according to the invention is mainly characterized by what is stated in the characterizing part of claim 3.

One further object of this invention are polymers containing (3R)-hydroxyacyl groups produced by the methods of this invention. In particular one object of this invention are PHAs (or compositions comprising PHAs), which have been produced by the methods of this invention and wherein PHAs consist of desired specified monomer chain lengths.

One still further object of this invention is a host cell or organism capable of producing PHAs.

More specifically, the host cell or organism capable of producing PHAs according to the invention is mainly characterized by what is stated in the characterizing part of claim 11.

One still further object of the present invention is a modified gene encoding MFE-2 protein, which comprises at least one gene region encoding a hydratase domain and at least one gene region encoding a dehydrogenase domain. The modification comprises that at least one genetic change has been made to the gene region encoding a dehydrogenase domain, which results in the enrichment of cellular (3R)-hydroxyacyl CoA esters of predetermined length, when the gene is introduced and expressed in a host cell.

More specifically, the gene according to the invention is mainly characterized by what is stated in the characterizing part of claim 15.

One still further object of the invention is a method for preparing a host cell capable of expressing a modified MFE-2 gene and a host cell or organism prepared by said method. The host may be selected from the group of bacteria, yeasts, other fungi, or higher eucaryotes, preferably plants.

The present invention results in various advantages. PHAs having specified monomer chain lengths and having desired physical and chemical properties can be produced, which was not possible by the prior art technology. Fewer purification steps are needed, because the combination of PHAs of various chains lengths can be controlled.

The production of PHAs is not restricted to the production of PHBs, which do not have advantageous properties for use in biodegradable plastics. PHAs with desired properties can be designed for the preparation of biodegradable plastics. In biomedical research monomeric 3-hydroxyacids with specified chain lengths can be used as reagents. No laborious and costly organic synthesis is needed.

In the following the invention will be described in more detail with the aid of a detailed description and a number of working examples. The embodiments are examples and they do not by any means limit the invention to these examples.

### BRIEF DESCRIPTION OF THE FIGURES

FIG. 1 depicts the joining of the 5-oxidation of fatty acids with the biosynthesis of polyhydroxyalkanoates.

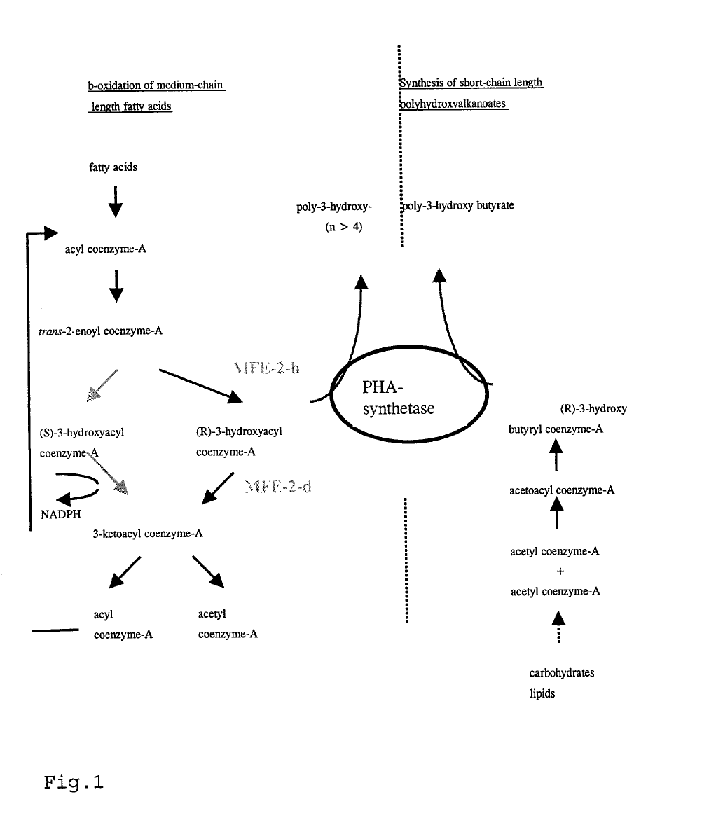

FIG. 2 shows the amino acid sequence of Saccharomyces cerevisiae MFE-2, (SWISSPROT Q02207) Length: 900 AA, MW 98703 Da (SEQ ID No. 15)

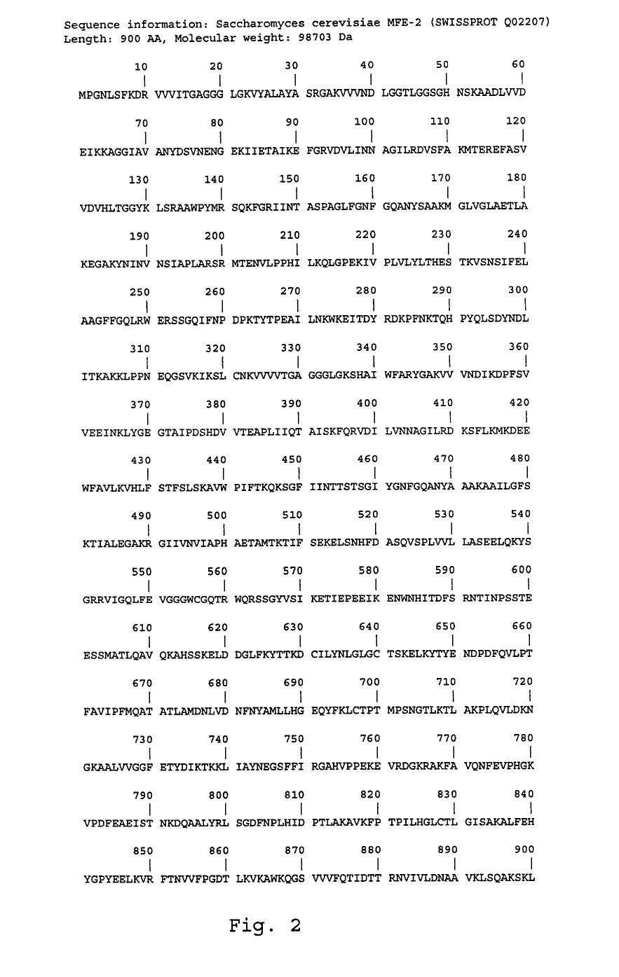

FIG. 3 shows the amino acid sequence of Saccharomyces cerevisiae MFE-2 mutant A (Gly to Ser mutation at position 16) Length: 900 AA, MW 98733 Da (SEQ ID No. 16)

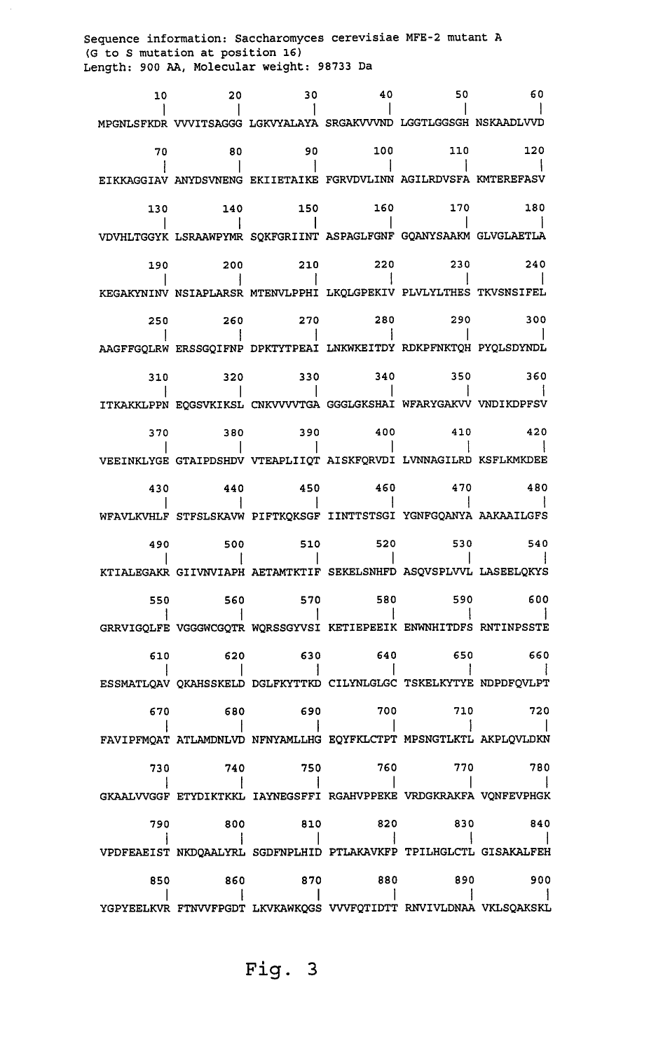

FIG. 4 shows the amino acid sequence of Saccharomyces cerevisiae MFE-2 mutant B (Gly to Ser mutation at position 329) Length: 900 AA, MW 98733 Da (SEQ ID No. 17)

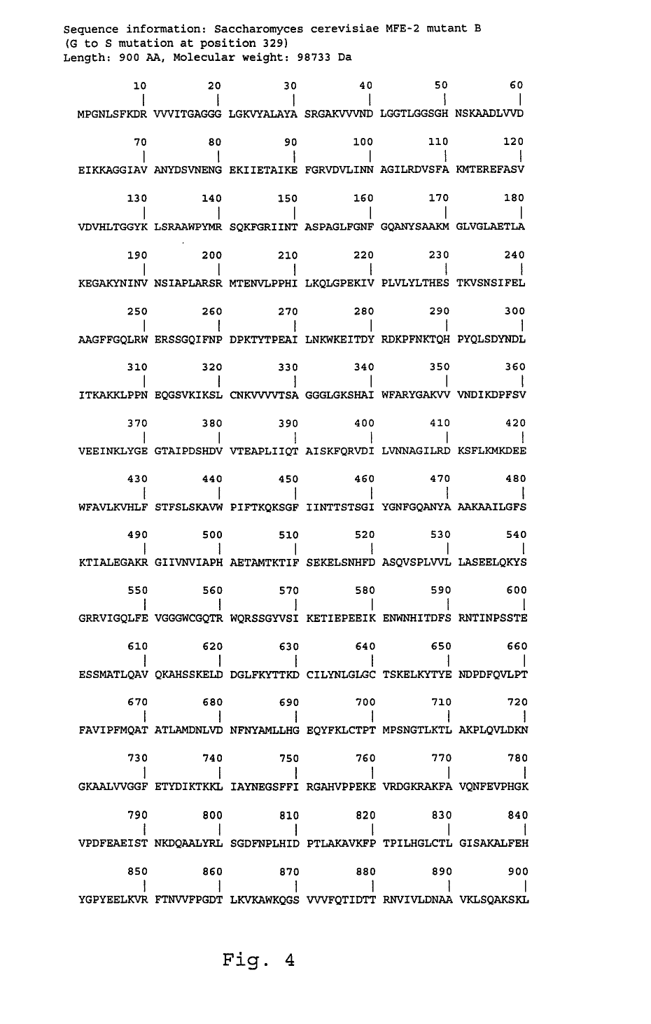

FIG. 5 shows the amino acid sequence of Saccharomyces cerevisiae MFE-2 mutant A and B (Gly to Ser mutation at position 16 and at position 329) Length: 900 AA, MW 98763 Da (SEQ ID No. 18)

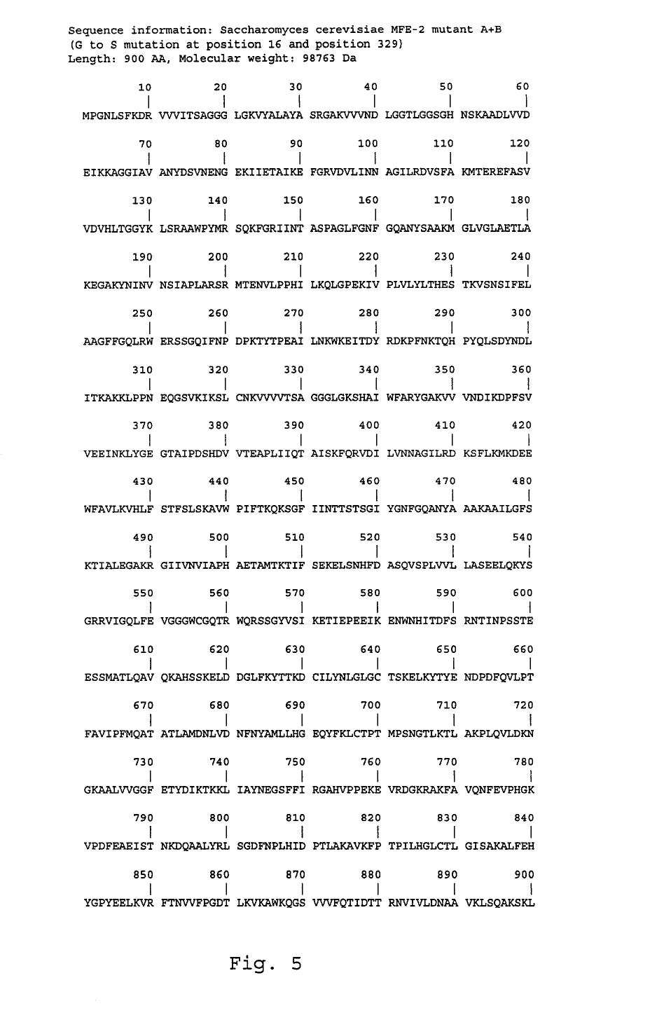

FIG. 6 shows the amino acid sequence of Candida tropicalis MFE-2 (SWISSPROT P22414) Length: 906 AA, MW 99469 Da (SEQ ID No. 19)

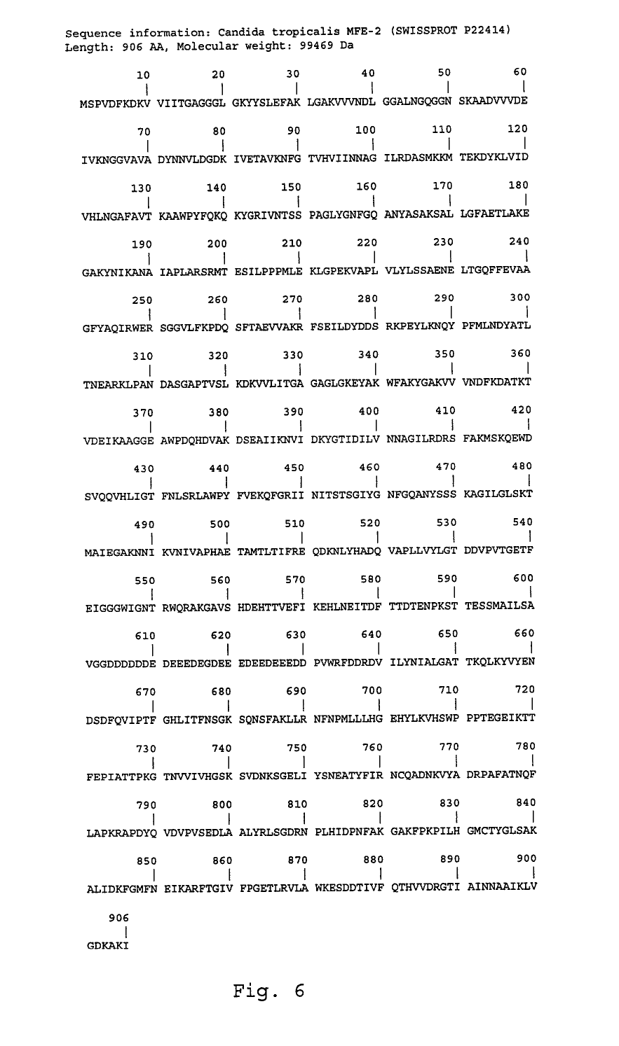

FIG. 7 shows the amino acid sequence of Candida tropicalis MFE-2 mutant A (Gly to Ser mutation at position 15) Length: 900 AA, MW 99499 Da (SEQ ID No. 20)

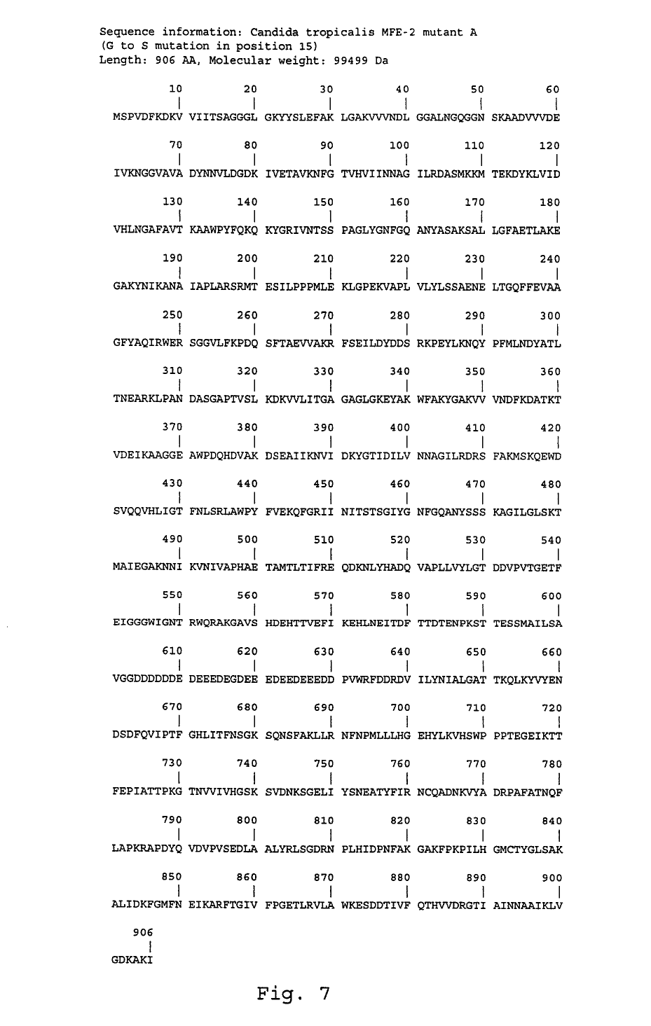

FIG. 8 shows the amino acid sequence of Candida tropicalis MFE-2 mutant B (Gly to Ser mutation at position 329) Length: 906 AA, MW 99499 Da (SEQ ID No. 21)

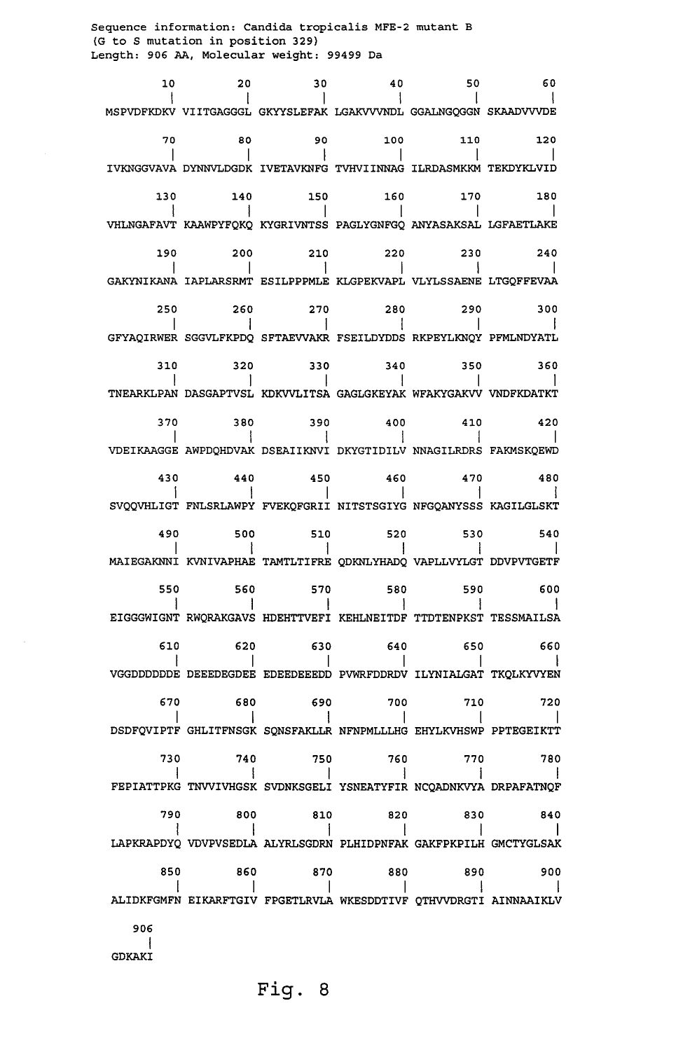

FIG. 9 shows the amino acid sequence of Candida tropicalis MFE-2 mutant A and B (Gly to Ser mutation at position 15 and at position 329) Length: 900 AA, MW 99529 Da (SEQ ID No. 22)

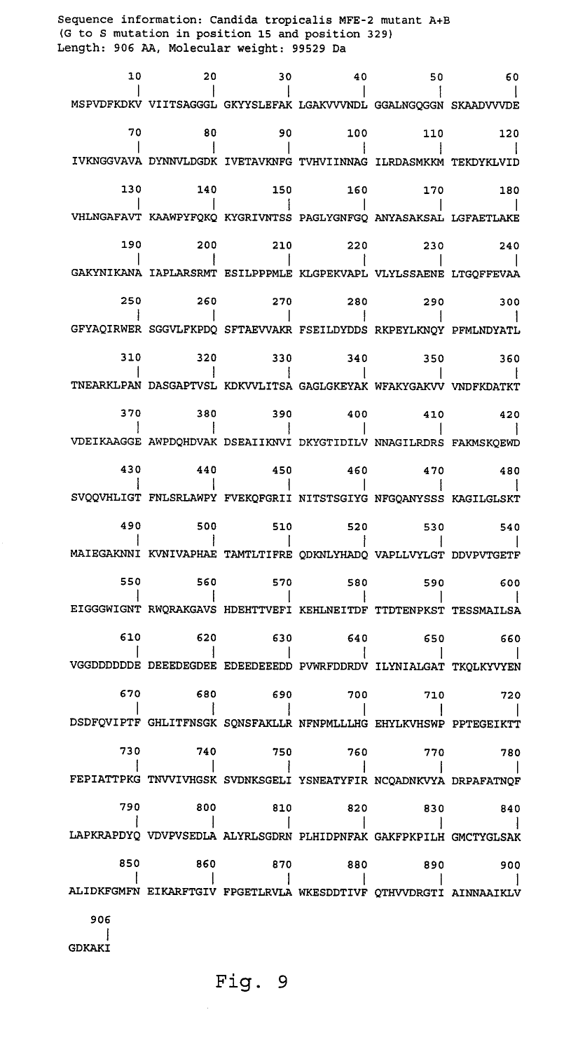

FIG. 10 shows the amino acid sequence of human MFE-2 (human 17-beta-hydroxysteroid dehydrogenase 4) (SWISSPROT P51659 ) Length: 736 AA, MW 79686 Da (SEQ ID No. 23)

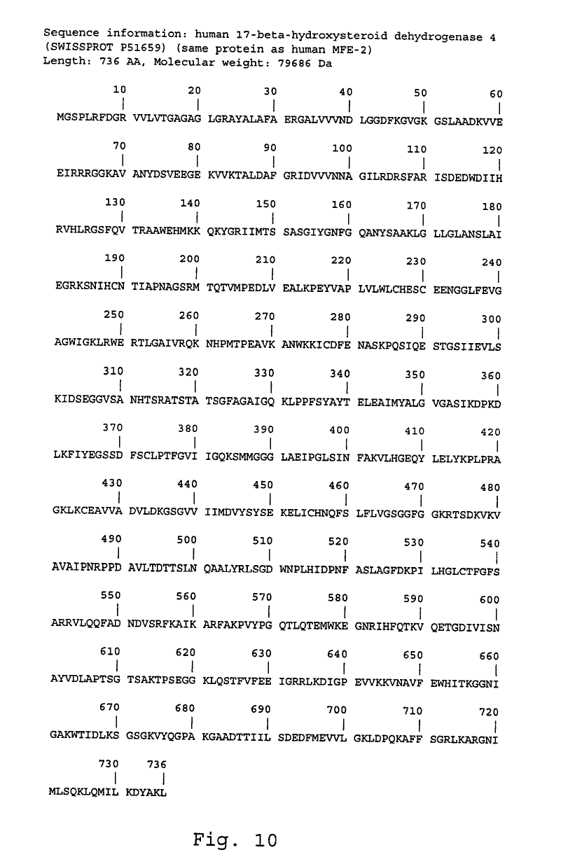

FIG. 11 shows the amino acid sequence of human MFE-2 mutant (Gly to Ser mutation at position 16) Length: 736 AA, MW 79686 Da (SEQ ID No. 24)

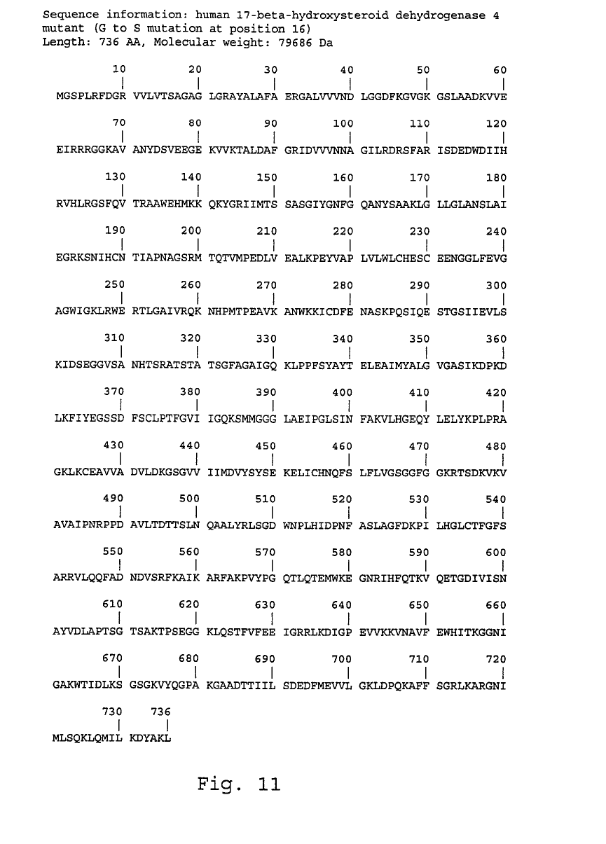

### DETAILED DESCRIPTION OF THE INVENTION

Definitions

MFE-2 is the abbreviation used here for a multifunctional 2-enoyl-CoA hydratase 2/(3R)-hydroxyacyl CoA dehydrogenase enzyme type 2 protein, which comprises at least one dehydrogenase domain and at least one hydratase domain. The term covers MFE-2 proteins from yeasts, other fungi and mammals as well as from other organisms possessing a protein with equivalent properties.

A gene encoding the MFE-2 protein refers to the DNA sequences encoding the MFE-2 enzyme in various organisms. The term “gene” refers to any DNA sequence encoding the MFE-2 enzyme, also to parts of genes, which are still capable of encoding MFE-2 enzyme. The DNA sequences may originate from organisms naturally expressing MFE-2 enzyme, such as from yeasts, other fungi, or from mammals including e.g. humans or rats, or the DNA sequences may be at least partly synthetically produced.

“Short chain length” (3R)-hydroxyacyl CoA esters stands here for (3R)-hydroxyacyl CoA esters of C2-C6, typically C4-C6.

“Medium chain length” (3R)-hydroxyacyl CoA esters stands here for (3R)-hydroxyacyl CoA esters of C6-C14, typically C8-C12.

“Long chain length” (3R)-hydroxyacyl CoA esters stands here for (3R)-hydroxyacyl CoA esters of C14-C20, typically C16.

By “genetic change” is meant here genetic methods such as deletions, substitutions, insertions and other mutations with which at least one dehydrogenase domain of MFE-2 protein encoding gene can be changed resulting in enrichment of cellular (3R)-hydroxyacyl-CoA esters of desired, predetermined length, when the modified gene is expressed in a chosen host cell or organism. The genetic change may comprise also that at least one hydratase domain or both at least one dehydrogenase domain and one hydratase domain is changed resulting in enrichment of cellular (3R)-hydroxyacyl-CoA esters of desired, predetermined length, when the modified gene is expressed in a chosen host cell or organism. Genetic change comprises preferably inactivation of a gene region, and refers to molecular biology methods, such as site-directed mutagenesis or deletion, which results in that the chosen gene region does not function. Both of these methods have been exemplified in the Examples. As described in the examples, the genetic modification resulting in the accumulation of medium and long chain-length 2-enoyl-CoA was obtained by mutating Gly to Ser at position 16 of the amino acid sequence of Saccharomyces cerevisiae MFE-2. The same result was obtained by mutating Gly to Ser at position 15 of Candida tropicalis MFE-2. The genetic modification resulting in the accumulation of short chain-length 2-enoyl-CoA was obtained by mutating Gly to Ser at position 329 of Saccharomyces cerevisiae MFE-2. The same position was mutated also in the MFE-2 of Candida tropicalis. In human MFE-2 encoding gene Gly was mutated to Ser at position 16. The genetic modifications are made by techniques well known in the art and a number of genes encoding MFE-2 have been cloned and characterized. The MFE-2 gene may originate from various different fungal genuses, such as from Candida or Saccharomyces genuses or the gene may originate from different mammals, such as from human, rat, mouse, or pig, and a person skilled in the art would know which position needs to be modified in order to obtain the desired result.

Van Grunsven et al (1998) has decribed MFE-2 deficiency in humans, which was caused by a mutation, where Gly 16 was mutated to Ser. (Qin et al. (1997a) describes the expression of a truncated rat MFE-2 encoding gene being devoid of (2R)-hydroxyacyl-CoA dehydrogenase activity. However, neither of these publications describe the role of modified MFE-2 in controlling the synthesis of PHA presursor molecules.

A “domain” refers to a gene region responsible for a specific function in a multifunctional enzyme. In the MFE-2 protein there are dehydrogenase domains and hydratase domains. In the yeast MFE-2 gene there are two dehydrogenase domains, A and B domains. Domain A has highest catalytic activity towards medium and long chain-length 2-enoyl-CoA, while domain B has the highest turn-over rate with short chain substrates. In mammalian MFE-2 there are only one dehydrogenase domain and one hydratase domain. In Table 1 the properties of MFE-2 from yeast and mammals and the domain specificities have been summarized.

TABLE 1
Properties of MFE-2 from yeast and mammals

| Variable | yeast | mammals |
|---|---|---|
| Dehydrogenase domain | two | one |
| Specificity | | |
| Domain A | medium and long chain | general |
| Domain B | short chain | — |
| Hydratase 2 domain | one | one |
| Specificity | general | medium chain |
| SC2-like domain | none | one |

According to a preferred embodiment of the invention the MFE-2 encoding gene originates from yeast. If the yeast domain having the highest catalytic activity towards medium and long chain-length 2-enoyl-CoA is inactivated and the gene is expressed in a chosen host, this results in the accumulation of medium and long chain-length 2-enoyl-CoA C8-C16 (typically C8-C12). In contrast, when the yeast domain having the highest turn-over rate with short chain substrates is inactivated, this results in the accumulation of short chain-length 2-enoyl-CoA C4-C8 (typically C6-C8).

If the gene originates from yeast and both of the dehydrogenase domains are inactivated and the gene is expressed in yeast, the yeast needs an alternative energy source. The peroxisomal multifunctional enzyme type 1 may be transferred to the yeast host resulting in the function of (3S)-hydroxyacyl-CoA specific β-oxidation pathway, which gives energy to the yeast. If the yeast host is a methylotrophic yeast, the energy may come from the oxidation of methanol. In plants the endogenous metabolism gives sufficient energy for the plant host.

If the mammalian dehydrogenase domain is inactivated, the oxidation of (3R)-hydroxyacyl-CoAs is blocked, and the chain length of the accumulating (3R)-hydroxyacyl intermediates is controlled by the kinetic properties of hydratase 2. The mammalian hydratase 2 is almost completely inactive toward short chain substrates and therefore the accumulating (3R)-hydroxyacyl-CoA esters are C8 or longer (up to C18).

In order to express the modified gene in a chosen host the gene should preferably be operably linked into regulatory sequences, in particular, a proper promoter functional in the host. The DNA construct comprising the modified gene and the regulatory sequences is then introduced into a host cell or organism. The DNA construct may be integrated into a vector capable of transferring the modified gene or DNA construct into the host cell.

The transformed host cell is grown under suitable culture conditions. If the host is a microbe host such as a bacterium or a yeast or other fungus host the growth medium may comprise glucose as carbon source allowing the microbe to synthesize fatty acids, which later go through the β-oxidation pathway. Alternatively fatty acids may be added to the growth medium. In plants fatty acids need not be added exogenously, because plants are capable of producing fatty acids through their endogenous metabolism.

If the host possesses a functional MFE-2 gene it can be removed or otherwise be inactivated before transferring and expressing the modified gene in the host.

If the host is a bacterial host it may possess a functional PHA synthetase gene. However, if the host is a yeast or other fungus or a higher eukaryotic host such as a plant, a functional PHA synthetase of bacterial origin may be transferred to the host. The PHA synthetase gene is transferred and preferably targeted to the peroxisomes of a plant as described by Mittendorf et al. (1998a and 1998b)

The bacterial host may be an E. coli, Bacillus or other bacterial host being suitable for expressing the modified MFE-2 gene. The fungal host may be an Aspergillus, Trichoderma, Saccharomyces, Candida or other host suitable for expressing the MFE-2 gene, as is known to a person skilled in the art.

The term “plant” encompasses any plant and progeny thereof. The term also encompasses parts of plants, including e.g. seeds, cuttings, buds, bulbs, somatic embryos etc. The plant may be a monocotyledonous plant or a dicotyledonous plant, in particular it may be a cultivated crop. Preferably it may be oilseed crop such as rapeseed, sunflower or soybean.

Oilseed crops are suggested as potential target plants for PHA production since these plants have naturally a high flux of carbon through acetyl-CoA intermediates. Rapeseed, sunflower and soybean have already been transformed with foreign genes, thus showing their potential for the technology (Poirier et al., 1995). PHA syntethase genes from Alcaligenes eutrophus and Pseudomonas aeruginosa have been transformed in cotton and Arabidopsis thaliana, respectively, resulting in both cases in PHB accumulation in the plant (John & Keller, 1996; Mittendorf et al., 1998a and WO 99/35278). These studies indicate that future PHA production in plants is a likely alternative.

For preparing the introduction of foreign genes into higher plants, a large number of cloning vectors are available which contain for example a replication signal for E. coli and a marker gene for the selection of transformed bacterial cells. Examples of such vectors are pBR322, pUC series, M13mp series.

A multitude of techniques are available for the introduction of DNA into a plant host cell. These techniques include the transformation of plant cells with T-DNAs using Agrobacterium tumefaciens or Agrobacterium rhizogenes as transformation agents, injection, the electroporation of DNA, the introduction of DNA using the bioballistic method and other possibilities. All these methods are well known in the art.

Methods to extract PHAs from different production hosts have been described (Mittendorf et al, 1998, Gerngross, 1999, U.S. Pat. No. 5,213,976). In the case of bacteria the cell wall must be broken using any of the everyday laboratory techniques, while plants can be physically ground either as fresh or frozen. The released PHA-containing biomass is then lyophilized, followed by sequential extraction of PHAs by methanol and chloroform. Final product can be concentrated by rotary evaporator commonly to at least 100 g of PHA per liter, and precipitated by methanol. These basic chemical handling techniques are straightforward to scale up.

(3R)-hydroxyacyl-CoA dehydrogenase activites of yeast MFE-2

The yeast MFE-2 protein has two N-terminal domains showing about 45% amino acid sequence similarity and belongs to the short-chain alcohol dehydrogenase/reductase superfamily. In the background of this invention was the interesting question from what physiological functions the two domains have or even whether both of them show enzymatic activities. To investigate the functions of these domains, here called A and B, Saccharomyces cerevisae fox-2 cells (devoid of endogenous MFE-2) were taken as a model system as disclosed in the Examples. Gly16 and Gly329 of the S. cerevisiae A and B domains, located in the predicted nucleotide binding sites, were mutated to serine and cloned into the yeast expression plasmid pYE352 (Hiltunen et al., 1992). In oleic acid medium, fox-2 cells transformed with pYE352::sMFE-2(a⁻) (mutation in the domain A) and pYE352::sMFE-2(b) (mutation in the domain B) grew slower than cells transformed with pYE352::sMFE-2 (wild type), whereas cells transformed with pYE352::sMFE-2(a⁻b⁻) (mutations in both A and B domains) failed to grow. Candida tropicalis MFE-2 with a deleted hydratase 2 domain [tMFE-2(h2Δ)] (truncated for the hydratase 2 domain), and mutational variants of the A and B domains tMFE-2(h2Δa⁻), tMFE-2(h2Δb⁻) and tMFE-2(h2Δa⁻b⁻)] were overexpressed and characterized. All proteins were dimers with similar secondary structure elements. Both wild-type domains were enzymatically active, but the B domain surprisingly showed the highest activity with short-chain and the A domain with medium- and long-chain (3R)-hydroxyacyl-CoA substrates.

An acquisition of two domains within a single polypeptide with different chain-length specificities is a novel strategy among lipid binding proteins to overcome the problems related to the metabolism of a large variety of substrates. Previously described strategies include gene duplication, which lead to the evolvement of separate enzymes, such as mammalian acyl-CoA dehydrogenases which are presented as several paralogues (Tanaka et al., 1990). Adaptation can also occur via the development of adaptive substrate binding pockets, as is formed in both 2-enoyl-CoA hydratase 1 (crotonase) and the acyl-CoA binding protein (Rosendal et al., 1993, Engel et al., 1996, Engel et al., 1998).

The experiments of yeast growing in liquid medium indicated that both domains A and B of MFE-2 are required for optimal growth of yeast cells on fatty acid as the sole carbon source. Yeast peroxisomal MFE-2 provides an intriguing example of one polypeptide which has acquired two enzymatically active dehydrogenase domains with different chain-length specificities. Thus it was found in the present invention that it provides a novel tool to control via site-directed mutagenesis the pool of (3R)-hydroxyacyl-CoA esters in transgenic organisms.

Mammalian MFE-2 can metabolize straight chain fatty acids under in vivo conditions and thus modulate 3-hydroxyacyl-CoA pool in a cell

As an example for mammalian MFE-2, the open reading frame of human MFE-2 cDNA was obtained from total RNA isolated from human fibroblasts by reverse transcription and amplified by PCR. The product was cloned behind oleic acid inducible yeast catalase A1 promotor in pYE352 vector and introduced into Saccharomyces cerevisiae fox-2 cells (devoid of endogenous MFE-2). The transformed strain regained the ability to grow on fatty acids as a carbon source. Although according to the current literature, the physiological role of mammalian MFE-2 is to participate in metabolism of α-methylacyl-CoA esters, the data showed unexpectedly that the mammalian MFE-2 can also metabolize straight chain acyl substrates under in vivo conditions. If a Gly16Ser mutation was introduced into the human MFE-2, by site-directed mutagenesis, the mutated variant did not complement the endogenous yeast MFE-2 in fox-2 cells.

β-Oxidation of fatty acids and PHA synthesis in transgenic higher eukaryotes

Many bacteria can synthesize medium chain length PHAs using intermediates of fatty acid β-oxidation. Because in bacteria only (3S)-hydroxyacyl-CoA dehydrogenase specific β-oxidation pathway has been described, it requires also the participation of NADPH-dependent 3-ketoacyl-CoA reductase. Recently, Poirier and coworkers targeted phaC1 from P. aeruginosa to peroxisomes of transgenic Arabidopsis and demonstrated an accumulation of inclusions in leafs containing PHA to approximately 4 mg per g of dry wt (Mittendorf et al., 1998). Even though PHAs are not natural metabolites of higher eukaryotes, this work demonstrates that plant cells can be used as bioreactors for producing PHAs. The transfer of PHA synthetase gene and the production of PHA in transgenic plants has been described in U.S. Pat. No. 5,650,555 and U.S. Pat. No. 5,750,848. Related technology is described also in U.S. Pat. No. 5,213,976.

The following examples are for illustration of the present invention and should not be construed as limiting the present invention in any manner.

### EXAMPLES

### Example 1

To study the functions of the two domains of yeast MFE-2, here called A and B, Saccharomyces cerevisae fox-2 cells (devoid of sMFE-2) (Hiltunen et al., 1992) were taken as a model system. The cDNA encoding sMFE-2 was obtained from S. cerevisiae genomic DNA by PCR with pfu high fidelity DNA polymerase (Stratagene, La Jolla, Calif., USA) using a 5′-primer, tctagagATG CCT GGA AAT TTA TCC TTC AAA G 3′ (SEQ ID No. 1), (and a 3′-primer, ctcgagaTTA TAG TTT AGA TTT TGC CTG CGA TAG 3′ (SEQ ID No. 2) containing XbaI and XhoI restriction sites, respectively (with lower case letters indicating mismatches to sMFE-2 gene). The amplified PCR-fragment was cloned into the pUC18 vector, and subsequently digested by XbaI and XhoI for cloning into pYE352 (Filppula et al 1995), resulting in pYE352::sMFE-2. pYE352::sMFE-2 was transformed into fox-2 cells (Gietz & Schiestl 1995) and selected on SD/Uracil plates.

Gly16 and Gly329 of the S. cerevisiae A and B domains, located in the predicted nucleotide binding sites and corresponding to Gly 16 which is mutated in the human MFE-2 deficiency (Van Grunsven et al 1998), were mutated to serine. Site-directed mutagenesis was performed according to the instructions of the QuikChange™ site-directed mutagenesis kit (Stratagene, La Jolla, Calif., USA). The sMFE-2 insert in pUC18 was taken as a template for generating pUC18::sMFE-2(a⁻) or pUC18::sMFE-2(b⁻). The primers designed for generating the Gly16Ser mutation in sMFE-2(a⁻) were 5′ GTT GTA ATC ACG TCT GCT GGA GGG GG 3′ (5′-primer) (SEQ ID No. 3) and 5′ CC CCC TCC AGC AGA CGT GAT TAC AAC 3′ (3′-primer) (SEQ ID No. 4). Primers, 5′ GTA GTA GTT ACG TCT GCA GGA GGT GGT C 3′ (5′-primer) (SEQ ID No. 5) and 5′ G ACC TCC TGC AGA CGT AAC TAC ATC 3′ (3′-primer) (SEQ ID No. 6) were used in generating the Gly329Ser mutation in sMFE-2(b⁻). pUC18::sMFE-2(a⁻) was used as a PCR template to obtain pUC18::sMFE-2(a⁻b⁻) with the primers designed for mutation Gly329Ser. All of the mutated DNA inserts were cloned into pYE352 (Hiltunen et al. 1992), resulting in pYE352::sMFE-2(a⁻), pYE352::sMFE-2(b⁻), and pYE352::sMFE-2(a⁻b⁻). In oleic acid medium, fox-2 cells transformed with pYE352::sMFE-2(a⁻) (mutation in the domain A; SEQ ID No. 16) and pYE352::sMFE-2(b⁻) (mutation in the domain B; SEQ ID No. 17) grew slower than cells transformed with pYE352::sMFE-2 (wild type), whereas cells transformed with pYE352::sMFE-2(a⁻b⁻) (mutations in both A and B domains; SEQ ID No. 18) failed to grow.

The in vitro characterization of the different variants of yeast MFE-2 was carried out with the protein and its variants from Candida tropicalis. The region of cDNA encoding amino acid residues 1-612 of C. tropicalis peroxisomal MFE-2 was amplified from the plasmid pMK22/HDE50 (Aitchison & Rachubinski 1990) by PCR with pfu polymerase, using the 5′-primer 5′ catATG TCT CCA GTT GAT TTT AAA 3′ (SEQ ID No. 7) and the 3′-primer 5′ ggatccttaTTC GTC TTC GTC ATC ATC A 3′ (SEQ ID No. 8) containing NdeI and BamHI restriction sites, respectively. The resulting 1839 base pair PCR fragment was subcloned into pUC18. Following digestion by NdeI and BamHI, the fragment was cloned into the pET3a expression vector (Novagen, Inc., Madison, Wis., USA) yielding pET3a::tMFE-2(h2Δ), and the nucleotide sequence encoding the dehydrogenase domains was verified. Site-directed mutagenesis was performed in a similar way as for the pYE352-sMFE-2 variants, using the tMFE-2 specific primers. By using pET3a::tMFE-2(h2Δ) as a PCR template, pET3a::tMFE-2(h2Δa⁻) was generated with the 5′-primer, 5′ GTG ATC ATT ACC AGT GCC GGT GGT G 3′ (SEQ ID No. 9) and the 3′-primer, 5′ C ACC ACC GGC ACT GGT AAT GAT CAC 3′ (SEQ ID No. 10), and pET3a::tMFE-2(h2Δb⁻) was generated with the 5′-primer, 5′ GTT TTG ATC ACC AGT GCC GGT GCT GG 3′ (SEQ ID No. 11) and the 3′-primer 5′ CC AGC ACC GGC ACT GGT GAT CAA AAC 3′ (SEQ ID No. 12). pET3a::tMFE-2(h2Δa⁻b⁻) was generated with the primers designed for tMFE-2(h2Δb⁻) using pET3a::tMFE-2(h2Δa⁻) as a template. The pET3a::tMFE-2(h2Δ) and its mutated variants were transformed into E. coli BL21 (DE3) plysS cells and expressed, the recombinant proteins were subsequently chromatographically purified from bacterial lysate to apparent homogeneity and characterized. All proteins were dimers (as shown by size exclusion chromatography) with similar secondary structure elements (as shown by far UV CD-spectropolarimetry).

Kinetic parameters were determined for the purified CtMFE-2(h2Δ) and its mutated variants toward oxidation of (3R)-hydroxyacyl-CoA 2 (Qin, et al. 1997a). Kinetic data were transformed to Lineweaver-Burk plots by using the GraFit computer software (Sigma Chemicals).

The tMFE-2(h2Δ) showed the highest catalytic efficiency (k_{cat}/Kₘ) with the substrate (3R)-hydroxydecanoyl-CoA (C10). The Kₘ value was lowest for the C10 substrate, being approximately one-fifth and one-tenth of the value of the C16 and C4 substrates, respectively. Interestingly, the (3R)-hydroxyacyl-CoA dehydrogenase activity of tMFE-2(h2Δ) broke into two different profiles when the mutated variants were analyzed. For tMFE-2(h2Δa⁻; SEQ ID No. 20), the catalytic constant (k_{cat}) of C4 was the same as for tMFE-2(h2Δ) (29±1 s−1 vs 31±2 s−1), whereas that of tMFE-2(h2Δb⁻; SEQ ID No. 21) was below the detection limit. The k_{cat} values of tMFE-2(h2Δa⁻) for C10 and C16 were 17±1 s−1 and 12±2 s−1. Interestingly, for tMFE-2(h2Δb⁻), the k_{cat} values were 33±2 s−1 and 36±6 s−1, suggesting that domain A contributes more than domain B in the metabolism of medium and long chain substrates. The activity of tMFE-2(h2Δa⁻b⁻; SEQ ID No. 22) toward the substrates tested was not detectable.

### Example 2

Mammalian MFE-2 can metabolize straight chain fatty acids under in vivo conditions and thus modulate the 3-hydroxyacyl-CoA pool in a cell.

As an example for mammalian MFE-2, the open reading frame (ORF) of human MFE-2 cDNA was obtained from total RNA isolated from human fibroblasts by reverse transcription, amplified by PCR using human MFE-2 specific primers the 5′-primer 5′-gagctctagaagATG GGC TCA CCC CTG AGG TTC GA-3′ (SEQ ID No. 13) and the 3′-primer 5′-ctcgagTCA GAG CTT GGC GTA GTC TTT AAG AA-3′ (SEQ ID No. 14) (lower case letters indicating mismatches to HuMFE-2 gene). The primers contained SacI and XhoI restriction sites for subsequent cloning into pUC18 vector using the Sure Clone Ligation kit (Amersham Pharmacia Biotech, Uppsala, Sweden). Following SacI and XhoI digestion, the HuMFE-2 insert (SEQ ID No. 23) was ligated into a similarly digested pYE352 behind the catalase A1 promoter (Filppula et al. 1995), resulting in pYE352::HuMFE-2. The Gly16Ser mutation was introduced into human MFE-2 (SEQ ID No. 24) (pYE352::HuMFE-2(dhΔ)), by site-directed mutagenesis following the procedure described for the yeast MFE-2 dehydrogenase variants. When pYE352::HuMFE-2 was introduced into S. cerevisiae fox-2 cells (devoid of endogenous MFE-2), the transformed strain regained the ability to grow on fatty acids. However, no utilisation of fatty acids were observed if the fox-2 cells were transformed with pYE352::HuMFE-2(dhΔ). When the enzyme activities of MFE-2 were measured in soluble extracts of transformed fox-2 cells, the hydratase 2 activity comparable to the wild type enzymes was observed, whereas the (3R)-hydroxyacyl-CoA dehydrogenase activity was erased by the Gly16Ser mutation.

### Literature

Aitchison, J. D., and Rachubinski, R. A. (1990) Curr. Genet. 17, 481-486

Anderson, A. J. & Dawes, E. A. (1990) Microbiol. Rev. 54: 450-472.

Dieuaide-Noubhani, M., Novikov, D., Baumgart, E., Vanhooren, J. C. T., Franzen, M., Goethals, M., Vandekerkhove, J., Van Veldhoven, P. P. & Mannerts, G. P. (1996) Eur. J. Biochem. 240, 660-666

Donadio, S., Staver, M. J., McAlpine, J. B., Swanson, S. J. & Katz, L. (1991) Science 252, 675-679

Engel, C. K., Mathieu, M., Zeelen, J. P., Hiltunen, J. K., and Wierenga, R. K. (1996) EMBO J. 15, 5135-5145

Engel, C. K., Kiema, T. R., Hiltunen, J. K., and Wierenga, R. K. (1998) J. Mol. Biol. 275, 847-859

Filppula, S. A., Sormunen, R. T., Hartig, A., Kunau, W. -H. & Hiltunen, J. K. (1995) J. Biol. Chem. 270, 27453-27457

Gerngross, T. U. (1999) Nature Biotechnology 17, 541-544

Gietz, R. D., and Schiestl, R. H. (1995) Methods Mol Cell Biol 5, 255-269.

Van Grunsven, E. G., van Berkel, E., Ijlst, L., Vreken, P., de Klerk, J. B. C., Adamski, J., Lemonde, H., Clayton, P. T., Cuebas, D. A. & R. J. A. (1998) Proc. Natl. Acad. Sci. USA 95, 2128-2133

Van Grunsven, E. G., van Berkel, E., Mooijer, P. A., Watkins, P. A., Moser, H. W., Suzuki, Y., Jiang, L. L., Hashimoto, T., Hoefler, G., Adamski, J. & Wanders, R. J. (1999) Am. J. Hum. Genet. 64, 99-107

Hiltunen, J. K., Wenzel, B., Beyer, A., Erdmann, R., Fosså, A. & Kunau, W. -H. (1992) J. Biol. Chem. 267, 6646-6653

John, M. E. & Keller, G. (1996) Proc. Natl. Acad. Sci. USA 93: 12768-12773.

Malila, L. H., Mäkelä, M. J., Jalonen, J. E., Latipää, P. M., Kunau, W. -H. & Hiltunen, J. K. (1993) J. Biol. Chem. 268, 21578-21585

Mittendorf, V., Robertson, E. J., Leecj, R. M., Krüiger, N., Steinbüchel, A. & Poirier, Y. (1998a) Proc. Natl. Acad. Sci. USA 95: 13397-13402.

Mittendorf, V., Krüger, N. Steinbüchel, A. and Poirier,Y. (1998b) In The Biochemical Principles and Mechanisms of Biosynthesis and Biodergadation of Polymers (Steinbüchel, A. ed.) Weinheim: Wiley-VCH, pp. 368-375.

Nawrath, C., Poirier, Y. & Somerville, C. (1994) Proc. Natl. Acad. Sci. USA 91: 12760-12764.

Poirier, Y., Dennis, D. E., Klomparens, K. & Somerville, C. (1992) Science 256: 520-523.

Poirier, Y., Nawrath, C. & Somerville, C. (1995) BIO/TECHNOLOGY 13: 142-150

Qin, Y. -M., Haapalainen, A. M., Conry, D., Cuebas, D. A., Hiltunen, J. K., and Novikov, D. K. (1997a) Biochem. J. 328, 377-382

Qin, Y. -M., Poutanen, M. H., Helander, H. M., Kvist, A. -P., Siivari, K. M., Schmitz, W., Conzelman E., Helmann, U. & Hiltunen, J. K. (1997b). Biochem. J., 321, 21-28

Steinbüchel, A. (1991) In: Novel Biomaterials from Biological Sources (D. Byrom, Ed.), MacMillan, New York.

Tanaka, K., Matsubara, Y., Indo, Y., Naito, E., Kraus, J. & Ozasa, H. (1990) Ann. N.Y. Acad. Sci. 321, 577-598.

Yao, J., Zhang, G., Wu, Q., Chen, G. -Q. & Zhang, R. (1999) Antonie van Leeuwenhoek 75(4), 345-349.

1. A method for controlling the production of cellular (3R)-hydroxyacyl CoA esters of predetermined length in a host cell or organism, characterized in that the method comprises the following steps:
introducing a gene encoding a multifunctional 2-enoyl-CoA hydratase 2/(3R)-hydroxyacyl CoA dehydrogenase enzyme type 2 protein (MFE-2) comprising at least one gene region encoding a hydratase domain and at least one gene region encoding a dehydrogenase domain, and wherein at least one genetic change has been made to the gene region encoding dehydrogenase domain, resulting in the enrichment of cellular (3R)-hydroxyacyl CoA esters of predetermined length, when the gene is introduced and expressed in a host cell oxidizing exogenous or endogenous fatty acids, or a DNA construct or a vector comprising the gene, into a host cell or organism; and
growing the host cell or organism under suitable growth conditions in the presence of a carbon source comprising fatty acids from an endogenous source or originating from exogenous additions, resulting in the enrichment of cellular (3R)-hydroxyacyl CoA esters of predetermined length.

2. A method for preparing a host cell or organism capable of producing PHAs, said host cell or organism expressing an endogenous or foreign gene or genes encoding (3R)-hydroxyacyl-CoA ester polymerizing enzyme or enzymes, characterized in that the method comprises the following steps:
introducing a gene encoding a multifunctional 2-enoyl-CoA hydratase 2/(3R)-hydroxyacyl CoA dehydrogenase enzyme type 2 protein (MFE-2), which comprises at least one gene region encoding a hydratase domain and at least one gene region encoding a dehydrogenase domain, wherein at least one genetic change has been made to the gene region encoding dehydrogenase domain, resulting in the enrichment of cellular (3R)-hydroxyacyl CoA esters of predetermined length, when the gene is introduced and expressed in a host cell oxidizing exogenous or endogenous fatty acids, or a DNA construct or a vector comprising the gene, into the host cell or organism; and
growing the host cell or organism under suitable growth conditions in the presence of a carbon source comprising fatty acids from an endogenous source or originating from exogenous additions.

3. A method for producing PHAs in a host cell or organism expressing endogenous or foreign gene or genes encoding (3R)-hydroxyacyl-CoA ester polymerizing enzyme or enzymes, characterized in that the method comprises the following steps:
introducing a gene encoding a multifunctional 2-enoyl-CoA hydratase 2/(3R)-hydroxyacyl CoA dehydrogenase enzyme type 2 protein (MFE-2) comprising at least one gene region encoding a hydratase domain and at least one gene region encoding a dehydrogenase domain, wherein at least one genetic change has been made to the gene region encoding dehydrogenase domain, resulting in the enrichment of cellular (3R)-hydroxyacyl CoA esters of predetermined length, when the gene is introduced and expressed in a host cell oxidizing exogenous or endogenous fatty acids or a DNA construct or a vector comprising the gene, into a host cell or organism;
growing the host cell or organism under suitable growth conditions in the presence of a carbon source comprising fatty acids from an endogenous source or originating from exogenous additions; and
recovering the PHAs or their hydrolysis products.

4. The method according to any one of claims 1-3, characterized in that the gene encoding the endogenous MFE-2 is inactivated in the host cell or organism.

5. The method according to any one of claims 1-3, characterized in that the gene encoding MFE-2 originates from yeast.

6. The method according to any one of claims 1-3, characterized in that the genetic change comprises inactivation of the gene region encoding the domain responsible for the oxidation of cellular short chain length (3R)-hydroxyacyl CoA esters resulting in the enrichment of cellular (3R)-hydroxyacyl CoA esters of C4-C8 chain length.

7. The method according to any one of claims 1-3, characterized in that the genetic change comprises inactivation of the gene region encoding the domain responsible for the oxidation of cellular (3R)-hydroxyacyl CoA esters of C8-C16 chain length resulting in the enrichment of cellular (3R)-hydroxyacyl CoA esters of C8-C16 chain length.

8. The method according to any one of claims 1-3, characterized in that the gene originates from mammals.

9. The method according to claim 8, characterized in that the change comprises inactivation of the gene region encoding the dehydrogenase domain resulting in the accumulation of cellular (3R)-hydroxyacyl CoA esters of C8- C18 chain length.

10. The method according to claim any one of claims 1-3, characterized in that the gene originates from yeast which encodes a multifunctional 2-enoyl-CoA hydratase 2/(3R)-hydroxyacyl CoA dehydrogenase enzyme type 2 protein comprising one gene region encoding hydratase domain and two gene regions encoding dehydrogenase domains, and wherein both gene regions encoding dehydrogenase domains are inactivated resulting in the enrichment of cellular (3R)-hydroxyacyl CoA esters of about C4- C20 length, when the gene is introduced and expressed in a host cell capable of β-oxidation of fatty acids.

11. A host cell or organism according to any one of claims 1-3, characterized in that the host cell is a bacterial, a yeast, a fungus or a plant cell.

12. The host cell or organism according to claim 11, characterized in that the host cell is a plant cell and that the method comprises growing the transformed plant cell into a transgenic plant.

13. A host cell or organism selected from the group consisting of a bacterial, a yeast, a fungus or a plant cell, characterized in that the host cell or organism is prepared by the method of claim 2.

14. A host cell or organism according to claim 13, characterized in that it is a plant cell, a plant or its progeny.

15. A gene encoding a multifunctional 2-enoyl-CoA hydratase 2/(3R)-hydroxyacyl CoA dehydrogenase enzyme type 2 protein (MFE-2) comprising at least one gene region encoding a hydratase domain and at least one gene region encoding a dehydrogenase domain, characterized in that at least one genetic change has been made to the gene region encoding dehydrogenase domain, resulting in the enrichment of cellular (3R)-hydroxyacyl CoA esters of predetermined length, when the gene is introduced and expressed in a host cell oxidizing exogenous or endogenous fatty acids.

16. The gene according to claim 15, characterized in that the gene originates from yeast.

17. The gene according to claim 16, characterized in that the genetic change comprises inactivation of the gene region encoding the domain responsible for the oxidation of cellular short chain length (3R)-hydroxyacyl CoA esters resulting in the enrichment of cellular (3R)-hydroxyacyl CoA esters of C4-C8 chain length.

18. The gene according to claim 17, characterized in that the gene encodes the amino acid sequence of SEQ ID NO. 17 or 21.

19. The gene according to claim 16, characterized in that the genetic change comprises inactivation of the gene region encoding the domain responsible for the oxidation of cellular (3R)-hydroxyacyl CoA esters of C8-C16 chain length resulting in the enrichment of cellular (3R)-hydroxyacyl CoA esters of C8-C16 chain length.

20. The gene according to claim 19, characterized in that the gene encodes the amino acid sequence of SEQ ID NO. 16 or 20.

21. The gene according to claim 15, characterized in that the gene originates from mammals.

22. The gene according to claim 21, characterized in that the genetic change comprises inactivation of the gene region encoding the dehydrogenase domain resulting in the accumulation of cellular (3R)-hydroxyacyl CoA esters of C8 -C18.

23. A yeast gene encoding a multifunctional 2-enoyl-CoA hydratase 2/(3R)-hydroxyacyl CoA dehydrogenase enzyme type 2 protein comprising one gene region encoding hydratase domain and two gene regions encoding dehydrogenase domains, characterized in that both gene regions encoding dehydrogenase domains are inactivated resulting in the enrichment of cellular (3R)-hydroxyacyl CoA esters of about C4-C20 length, when the gene is introduced and expressed in a host cell capable of β-oxidation of fatty acids.

24. The gene according to claim 23, characterized in that the gene encodes the amino acid sequence of SEQ ID NO. 18 or 22.

25. A DNA construct comprising the gene of claim 15.

26. A vector comprising the gene of claim 15.

27. A host cell comprising the gene of claim 15.

28. The host cell of claim 27, characterized in that the cell is a bacterial, yeast, fungus or a plant cell.

29. A plant or its progeny comprising the cells of claim 28.

30. A multifunctional enzyme encoded by the gene of claim 15.

31. A method for preparing a host cell or organism capable of expressing a modified 2-enoyl-CoA hydratase 2/(3R)-hydroxyacyl CoA dehydrogenase enzyme type 2 protein, characterized in that the method comprises the following steps:
introducing the gene of claim 15 into a host cell or organism; and
growing the host cell under suitable conditions.

32. A host cell or organism prepared by the method of claim 31, characterized in that it is selected from the group consisting of host cells or organisms belonging to bacteria, yeast, fungus or plants.


SEQUENCE LISTING (24 sequences)

SEQ ID NO: 1 - DNA, 33 bp, Artificial Sequence
  Description of Artificial Sequenceprimer
tctagaagatgcctggaaatttatccttcaaag

SEQ ID NO: 2 - DNA, 34 bp, Artificial Sequence
  Description of Artificial Sequenceprimer
ctcgagattatagtttagattttgcctgcgatag

SEQ ID NO: 3 - DNA, 26 bp, Artificial Sequence
  Description of Artificial Sequenceprimer
gttgtaatcacgtctgctggaggggg

SEQ ID NO: 4 - DNA, 26 bp, Artificial Sequence
  Description of Artificial Sequenceprimer
ccccctccagcagacgtgattacaac

SEQ ID NO: 5 - DNA, 28 bp, Artificial Sequence
  Description of Artificial Sequenceprimer
gtagtagttacgtctgcaggaggtggtc

SEQ ID NO: 6 - DNA, 28 bp, Artificial Sequence
  Description of Artificial Sequenceprimer
gtagtagttacgtctgcaggaggtggtc

SEQ ID NO: 7 - DNA, 24 bp, Artificial Sequence
  Description of Artificial Sequenceprimer
catatgtctccagttgattttaaa

SEQ ID NO: 8 - DNA, 28 bp, Artificial Sequence
  Description of Artificial Sequenceprimer
ggatccttattcgtcttcgtcatcatca

SEQ ID NO: 9 - DNA, 25 bp, Artificial Sequence
  Description of Artificial Sequenceprimer
gtgatcattaccagtgccggtggtg

SEQ ID NO: 10 - DNA, 25 bp, Artificial Sequence
  Description of Artificial Sequenceprimer
caccaccggcactggtaatgatcac

SEQ ID NO: 11 - DNA, 26 bp, Artificial Sequence
  Description of Artificial Sequenceprimer
gttttgatcaccagtgccggtgctgg

SEQ ID NO: 12 - DNA, 26 bp, Artificial Sequence
  Description of Artificial Sequenceprimer
ccagcaccggcactggtgatcaaaac

SEQ ID NO: 13 - DNA, 35 bp, Artificial Sequence
  Description of Artificial Sequenceprimer
gagctctagaagatgggctcacccctgaggttcga

SEQ ID NO: 14 - DNA, 32 bp, Artificial Sequence
  Description of Artificial Sequenceprimer
ctcgagtcagagcttggcgtagtctttaagaa

SEQ ID NO: 15 - protein, 900 aa, Saccharomyces cerevisiae
MPGNLSFKDRVVVITGAGGGLGKVYALAYASRGAKVVVNDLGGTLGGSGHNSKAADLVVDEIKKAGGIAVANYDSVNENGEKIIETAIKEFGRVDVLINNAGILRDVSFAKMTEREFASVVDVHLTGGYKLSRAAWPYMRSQKFGRIINTASPAGLFGNFGQANYSAAKMGLVGLAETLAKEGAKYNINVNSIAPLARSRMTENVLPPHILKQLGPEKIVPLVLYLTHESTKVSNSIFELAAGFFGQLRWERSSGQIFNPDPKTYTPEAILNKWKEITDYRDKPFNKTQHPYQLSDYNDLITKAKKLPPNEQGSVKIKSLCNKVVVVTGAGGGLGKSHAIWFARYGAKVVVNDIKDPFSVVEEINKLYGEGTAIPDSHDVVTEAPLIIQTAISKFQRVDILVNNAGILRDKSFLKMKDEEWFAVLKVHLFSTFSLSKAVWPIFTKQKSGFIINTTSTSGIYGNFGQANYAAAKAAILGFSKTIALEGAKRGIIVNVIAPHAETAMTKTIFSEKELSNHFDASQVSPLVVLLASEELQKYSGRRVIGQLFEVGGGWCGQTRWQRSSGYVSIKETIEPEEIKENWNHITDFSRNTINPSSTEESSMATLQAVQKAHSSKELDDGLFKYTTKDCILYNLGLGCTSKELKYTYENDPDFQVLPTFAVIPFMQATATLAMDNLVDNFNYAMLLHGEQYFKLCTPTMPSNGTLKTLAKPLQVLDKNGKAALVVGGFETYDIKTKKLIAYNEGSFFIRGAHVPPEKEVRDGKRAKFAVQNFEVPHGKVPDFEAEISTNKDQAALYRLSGDFNPLHIDPTLAKAVKFPTPILHGLCTLGISAKALFEHYGPYEELKVRFTNVVFPGDTLKVKAWKQGSVVVFQTIDTTRNVIVLDNAAVKLSQAKSKL

SEQ ID NO: 16 - protein, 900 aa, Saccharomyces cerevisiae
MPGNLSFKDRVVVITSAGGGLGKVYALAYASRGAKVVVNDLGGTLGGSGHNSKAADLVVDEIKKAGGIAVANYDSVNENGEKIIETAIKEFGRVDVLINNAGILRDVSFAKMTEREFASVVDVHLTGGYKLSRAAWPYMRSQKFGRIINTASPAGLFGNFGQANYSAAKMGLVGLAETLAKEGAKYNINVNSIAPLARSRMTENVLPPHILKQLGPEKIVPLVLYLTHESTKVSNSIFELAAGFFGQLRWERSSGQIFNPDPKTYTPEAILNKWKEITDYRDKPFNKTQHPYQLSDYNDLITKAKKLPPNEQGSVKIKSLCNKVVVVTGAGGGLGKSHAIWFARYGAKVVVNDIKDPFSVVEEINKLYGEGTAIPDSHDVVTEAPLIIQTAISKFQRVDILVNNAGILRDKSFLKMKDEEWFAVLKVHLFSTFSLSKAVWPIFTKQKSGFIINTTSTSGIYGNFGQANYAAAKAAILGFSKTIALEGAKRGIIVNVIAPHAETAMTKTIFSEKELSNHFDASQVSPLVVLLASEELQKYSGRRVIGQLFEVGGGWCGQTRWQRSSGYVSIKETIEPEEIKENWNHITDFSRNTINPSSTEESSMATLQAVQKAHSSKELDDGLFKYTTKDCILYNLGLGCTSKELKYTYENDPDFQVLPTFAVIPFMQATATLAMDNLVDNFNYAMLLHGEQYFKLCTPTMPSNGTLKTLAKPLQVLDKNGKAALVVGGFETYDIKTKKLIAYNEGSFFIRGAHVPPEKEVRDGKRAKFAVQNFEVPHGKVPDFEAEISTNKDQAALYRLSGDFNPLHIDPTLAKAVKFPTPILHGLCTLGISAKALFEHYGPYEELKVRFTNVVFPGDTLKVKAWKQGSVVVFQTIDTTRNVIVLDNAAVKLSQAKSKL

SEQ ID NO: 17 - protein, 900 aa, Saccharomyces cerevisiae
MPGNLSFKDRVVVITGAGGGLGKVYALAYASRGAKVVVNDLGGTLGGSGHNSKAADLVVDEIKKAGGIAVANYDSVNENGEKIIETAIKEFGRVDVLINNAGILRDVSFAKMTEREFASVVDVHLTGGYKLSRAAWPYMRSQKFGRIINTASPAGLFGNFGQANYSAAKMGLVGLAETLAKEGAKYNINVNSIAPLARSRMTENVLPPHILKQLGPEKIVPLVLYLTHESTKVSNSIFELAAGFFGQLRWERSSGQIFNPDPKTYTPEAILNKWKEITDYRDKPFNKTQHPYQLSDYNDLITKAKKLPPNEQGSVKIKSLCNKVVVVTSAGGGLGKSHAIWFARYGAKVVVNDIKDPFSVVEEINKLYGEGTAIPDSHDVVTEAPLIIQTAISKFQRVDILVNNAGILRDKSFLKMKDEEWFAVLKVHLFSTFSLSKAVWPIFTKQKSGFIINTTSTSGIYGNFGQANYAAAKAAILGFSKTIALEGAKRGIIVNVIAPHAETAMTKTIFSEKELSNHFDASQVSPLVVLLASEELQKYSGRRVIGQLFEVGGGWCGQTRWQRSSGYVSIKETIEPEEIKENWNHITDFSRNTINPSSTEESSMATLQAVQKAHSSKELDDGLFKYTTKDCILYNLGLGCTSKELKYTYENDPDFQVLPTFAVIPFMQATATLAMDNLVDNFNYAMLLHGEQYFKLCTPTMPSNGTLKTLAKPLQVLDKNGKAALVVGGFETYDIKTKKLIAYNEGSFFIRGAHVPPEKEVRDGKRAKFAVQNFEVPHGKVPDFEAEISTNKDQAALYRLSGDFNPLHIDPTLAKAVKFPTPILHGLCTLGISAKALFEHYGPYEELKVRFTNVVFPGDTLKVKAWKQGSVVVFQTIDTTRNVIVLDNAAVKLSQAKSKL

SEQ ID NO: 18 - protein, 900 aa, Saccharomyces cerevisiae
MPGNLSFKDRVVVITSAGGGLGKVYALAYASRGAKVVVNDLGGTLGGSGHNSKAADLVVDEIKKAGGIAVANYDSVNENGEKIIETAIKEFGRVDVLINNAGILRDVSFAKMTEREFASVVDVHLTGGYKLSRAAWPYMRSQKFGRIINTASPAGLFGNFGQANYSAAKMGLVGLAETLAKEGAKYNINVNSIAPLARSRMTENVLPPHILKQLGPEKIVPLVLYLTHESTKVSNSIFELAAGFFGQLRWERSSGQIFNPDPKTYTPEAILNKWKEITDYRDKPFNKTQHPYQLSDYNDLITKAKKLPPNEQGSVKIKSLCNKVVVVTSAGGGLGKSHAIWFARYGAKVVVNDIKDPFSVVEEINKLYGEGTAIPDSHDVVTEAPLIIQTAISKFQRVDILVNNAGILRDKSFLKMKDEEWFAVLKVHLFSTFSLSKAVWPIFTKQKSGFIINTTSTSGIYGNFGQANYAAAKAAILGFSKTIALEGAKRGIIVNVIAPHAETAMTKTIFSEKELSNHFDASQVSPLVVLLASEELQKYSGRRVIGQLFEVGGGWCGQTRWQRSSGYVSIKETIEPEEIKENWNHITDFSRNTINPSSTEESSMATLQAVQKAHSSKELDDGLFKYTTKDCILYNLGLGCTSKELKYTYENDPDFQVLPTFAVIPFMQATATLAMDNLVDNFNYAMLLHGEQYFKLCTPTMPSNGTLKTLAKPLQVLDKNGKAALVVGGFETYDIKTKKLIAYNEGSFFIRGAHVPPEKEVRDGKRAKFAVQNFEVPHGKVPDFEAEISTNKDQAALYRLSGDFNPLHIDPTLAKAVKFPTPILHGLCTLGISAKALFEHYGPYEELKVRFTNVVFPGDTLKVKAWKQGSVVVFQTIDTTRNVIVLDNAAVKLSQAKSKL

SEQ ID NO: 19 - protein, 906 aa, Candida tropicalis
MSPVDFKDKVVIITGAGGGLGKYYSLEFAKLGAKVVVNDLGGALNGQGGNSKAADVVVDEIVKNGGVAVADYNNVLDGDKIVETAVKNFGTVHVIINNAGILRDASMKKMTEKDYKLVIDVHLNGAFAVTKAAWPYFQKQKYGRIVNTSSPAGLYGNFGQANYASAKSALLGFAETLAKEGAKYNIKANAIAPLARSRMTESILPPPMLEKLGPEKVAPLVLYLSSAENELTGQFFEVAAGFYAQIRWERSGGVLFKPDQSFTAEVVAKRFSEILDYDDSRKPEYLKNQYPFMLNDYATLTNEARKLPANDASGAPTVSLKDKVVLITGAGAGLGKEYAKWFAKYGAKVVVNDFKDATKTVDEIKAAGGEAWPDQHDVAKDSEAIIKNVIDKYGTIDILVNNAGILRDRSFAKMSKQEWDSVQQVHLIGTFNLSRLAWPYFVEKQFGRIINITSTSGIYGNFGQANYSSSKAGILGLSKTMAIEGAKNNIKVNIVAPHAETAMTLTIFREQDKNLYHADQVAPLLVYLGTDDVPVTGETFEIGGGWIGNTRWQRAKGAVSHDEHTTVEFIKEHLNEITDFTTDTENPKSTTESSMAILSAVGGDDDDDDEDEEEDEGDEEEDEEDEEEDDPVWRFDDRDVILYNIALGATTKQLKYVYENDSDFQVIPTFGHLITFNSGKSQNSFAKLLRNFNPMLLLHGEHYLKVHSWPPPTEGEIKTTFEPIATTPKGTNVVIVHGSKSVDNKSGELIYSNEATYFIRNCQADNKVYADRPAFATNQFLAPKRAPDYQVDVPVSEDLAALYRLSGDRNPLHIDPNFAKGAKFPKPILHGMCTYGLSAKALIDKFGMFNEIKARFTGIVFPGETLRVLAWKESDDTIVFQTHVVDRGTIAINNAAIKLVGDKAKI

SEQ ID NO: 20 - protein, 906 aa, Candida tropicalis
MSPVDFKDKVVIITSAGGGLGKYYSLEFAKLGAKVVVNDLGGALNGQGGNSKAADVVVDEIVKNGGVAVADYNNVLDGDKIVETAVKNFGTVHVIINNAGILRDASMKKMTEKDYKLVIDVHLNGAFAVTKAAWPYFQKQKYGRIVNTSSPAGLYGNFGQANYASAKSALLGFAETLAKEGAKYNIKANAIAPLARSRMTESILPPPMLEKLGPEKVAPLVLYLSSAENELTGQFFEVAAGFYAQIRWERSGGVLFKPDQSFTAEVVAKRFSEILDYDDSRKPEYLKNQYPFMLNDYATLTNEARKLPANDASGAPTVSLKDKVVLITGAGAGLGKEYAKWFAKYGAKVVVNDFKDATKTVDEIKAAGGEAWPDQHDVAKDSEAIIKNVIDKYGTIDILVNNAGILRDRSFAKMSKQEWDSVQQVHLIGTFNLSRLAWPYFVEKQFGRIINITSTSGIYGNFGQANYSSSKAGILGLSKTMAIEGAKNNIKVNIVAPHAETAMTLTIFREQDKNLYHADQVAPLLVYLGTDDVPVTGETFEIGGGWIGNTRWQRAKGAVSHDEHTTVEFIKEHLNEITDFTTDTENPKSTTESSMAILSAVGGDDDDDDEDEEEDEGDEEEDEEDEEEDDPVWRFDDRDVILYNIALGATTKQLKYVYENDSDFQVIPTFGHLITFNSGKSQNSFAKLLRNFNPMLLLHGEHYLKVHSWPPPTEGEIKTTFEPIATTPKGTNVVIVHGSKSVDNKSGELIYSNEATYFIRNCQADNKVYADRPAFATNQFLAPKRAPDYQVDVPVSEDLAALYRLSGDRNPLHIDPNFAKGAKFPKPILHGMCTYGLSAKALIDKFGMFNEIKARFTGIVFPGETLRVLAWKESDDTIVFQTHVVDRGTIAINNAAIKLVGDKAKI

SEQ ID NO: 21 - protein, 906 aa, Candida tropicalis
MSPVDFKDKVVIITGAGGGLGKYYSLEFAKLGAKVVVNDLGGALNGQGGNSKAADVVVDEIVKNGGVAVADYNNVLDGDKIVETAVKNFGTVHVIINNAGILRDASMKKMTEKDYKLVIDVHLNGAFAVTKAAWPYFQKQKYGRIVNTSSPAGLYGNFGQANYASAKSALLGFAETLAKEGAKYNIKANAIAPLARSRMTESILPPPMLEKLGPEKVAPLVLYLSSAENELTGQFFEVAAGFYAQIRWERSGGVLFKPDQSFTAEVVAKRFSEILDYDDSRKPEYLKNQYPFMLNDYATLTNEARKLPANDASGAPTVSLKDKVVLITSAGAGLGKEYAKWFAKYGAKVVVNDFKDATKTVDEIKAAGGEAWPDQHDVAKDSEAIIKNVIDKYGTIDILVNNAGILRDRSFAKMSKQEWDSVQQVHLIGTFNLSRLAWPYFVEKQFGRIINITSTSGIYGNFGQANYSSSKAGILGLSKTMAIEGAKNNIKVNIVAPHAETAMTLTIFREQDKNLYHADQVAPLLVYLGTDDVPVTGETFEIGGGWIGNTRWQRAKGAVSHDEHTTVEFIKEHLNEITDFTTDTENPKSTTESSMAILSAVGGDDDDDDEDEEEDEGDEEEDEEDEEEDDPVWRFDDRDVILYNIALGATTKQLKYVYENDSDFQVIPTFGHLITFNSGKSQNSFAKLLRNFNPMLLLHGEHYLKVHSWPPPTEGEIKTTFEPIATTPKGTNVVIVHGSKSVDNKSGELIYSNEATYFIRNCQADNKVYADRPAFATNQFLAPKRAPDYQVDVPVSEDLAALYRLSGDRNPLHIDPNFAKGAKFPKPILHGMCTYGLSAKALIDKFGMFNEIKARFTGIVFPGETLRVLAWKESDDTIVFQTHVVDRGTIAINNAAIKLVGDKAKI

SEQ ID NO: 22 - protein, 906 aa, Candida tropicalis
MSPVDFKDKVVIITSAGGGLGKYYSLEFAKLGAKVVVNDLGGALNGQGGNSKAADVVVDEIVKNGGVAVADYNNVLDGDKIVETAVKNFGTVHVIINNAGILRDASMKKMTEKDYKLVIDVHLNGAFAVTKAAWPYFQKQKYGRIVNTSSPAGLYGNFGQANYASAKSALLGFAETLAKEGAKYNIKANAIAPLARSRMTESILPPPMLEKLGPEKVAPLVLYLSSAENELTGQFFEVAAGFYAQIRWERSGGVLFKPDQSFTAEVVAKRFSEILDYDDSRKPEYLKNQYPFMLNDYATLTNEARKLPANDASGAPTVSLKDKVVLITSAGAGLGKEYAKWFAKYGAKVVVNDFKDATKTVDEIKAAGGEAWPDQHDVAKDSEAIIKNVIDKYGTIDILVNNAGILRDRSFAKMSKQEWDSVQQVHLIGTFNLSRLAWPYFVEKQFGRIINITSTSGIYGNFGQANYSSSKAGILGLSKTMAIEGAKNNIKVNIVAPHAETAMTLTIFREQDKNLYHADQVAPLLVYLGTDDVPVTGETFEIGGGWIGNTRWQRAKGAVSHDEHTTVEFIKEHLNEITDFTTDTENPKSTTESSMAILSAVGGDDDDDDEDEEEDEGDEEEDEEDEEEDDPVWRFDDRDVILYNIALGATTKQLKYVYENDSDFQVIPTFGHLITFNSGKSQNSFAKLLRNFNPMLLLHGEHYLKVHSWPPPTEGEIKTTFEPIATTPKGTNVVIVHGSKSVDNKSGELIYSNEATYFIRNCQADNKVYADRPAFATNQFLAPKRAPDYQVDVPVSEDLAALYRLSGDRNPLHIDPNFAKGAKFPKPILHGMCTYGLSAKALIDKFGMFNEIKARFTGIVFPGETLRVLAWKESDDTIVFQTHVVDRGTIAINNAAIKLVGDKAKI

SEQ ID NO: 23 - protein, 736 aa, Homo sapiens
MGSPLRFDGRVVLVTGAGAGLGRAYALAFAERGALVVVNDLGGDFKGVGKGSLAADKVVEEIRRRGGKAVANYDSVEEGEKVVKTALDAFGRIDVVVNNAGILRDRSFARISDEDWDIIHRVHLRGSFQVTRAAWEHMKKQKYGRIIMTSSASGIYGNFGQANYSAAKLGLLGLANSLAIEGRKSNIHCNTIAPNAGSRMTQTVMPEDLVEALKPEYVAPLVLWLCHESCEENGGLFEVGAGWIGKLRWERTLGAIVRQKNHPMTPEAVKANWKKICDFENASKPQSIQESTGSIIEVLSKIDSEGGVSANHTSRATSTATSGFAGAIGQKLPPFSYAYTELEAIMYALGVGASIKDPKDLKFIYEGSSDFSCLPTFGVIIGQKSMMGGGLAEIPGLSINFAKVLHGEQYLELYKPLPRAGKLKCEAVVADVLDKGSGVVIIMDVYSYSEKELICHNQFSLFLVGSGGFGGKRTSDKVKVAVAIPNRPPDAVLTDTTSLNQAALYRLSGDWNPLHIDPNFASLAGFDKPILHGLCTFGFSARRVLQQFADNDVSRFKAIKARFAKPVYPGQTLQTEMWKEGNRIHFQTKVQETGDIVISNAYVDLAPTSGTSAKTPSEGGKLQSTFVFEEIGRRLKDIGPEVVKKVNAVFEWHITKGGNIGAKWTIDLKSGSGKVYQGPAKGAADTTIILSDEDFMEVVLGKLDPQKAFFSGRLKARGNIMLSQKLQMILKDYAKL

SEQ ID NO: 24 - protein, 736 aa, Homo sapiens
MGSPLRFDGRVVLVTSAGAGLGRAYALAFAERGALVVVNDLGGDFKGVGKGSLAADKVVEEIRRRGGKAVANYDSVEEGEKVVKTALDAFGRIDVVVNNAGILRDRSFARISDEDWDIIHRVHLRGSFQVTRAAWEHMKKQKYGRIIMTSSASGIYGNFGQANYSAAKLGLLGLANSLAIEGRKSNIHCNTIAPNAGSRMTQTVMPEDLVEALKPEYVAPLVLWLCHESCEENGGLFEVGAGWIGKLRWERTLGAIVRQKNHPMTPEAVKANWKKICDFENASKPQSIQESTGSIIEVLSKIDSEGGVSANHTSRATSTATSGFAGAIGQKLPPFSYAYTELEAIMYALGVGASIKDPKDLKFIYEGSSDFSCLPTFGVIIGQKSMMGGGLAEIPGLSINFAKVLHGEQYLELYKPLPRAGKLKCEAVVADVLDKGSGVVIIMDVYSYSEKELICHNQFSLFLVGSGGFGGKRTSDKVKVAVAIPNRPPDAVLTDTTSLNQAALYRLSGDWNPLHIDPNFASLAGFDKPILHGLCTFGFSARRVLQQFADNDVSRFKAIKARFAKPVYPGQTLQTEMWKEGNRIHFQTKVQETGDIVISNAYVDLAPTSGTSAKTPSEGGKLQSTFVFEEIGRRLKDIGPEVVKKVNAVFEWHITKGGNIGAKWTIDLKSGSGKVYQGPAKGAADTTIILSDEDFMEVVLGKLDPQKAFFSGRLKARGNIMLSQKLQMILKDYAKL